(19) United States
(12) Patent Application Publication
(10) Pub. No.: US 2005/0112671 A1
(43) Pub. Date: May 26, 2005

(54) FRET efficiency methods

(75) Inventors: Anthony M. Maletta, Woburn, MA (US); Ian McBee, Woburn, MA (US); Xiaojian (David) Zhao, Westford, MA (US); Martin Fuchs, Uxbridge, MA (US)

Correspondence Address: WOLF GREENFIELD & SACKS, PC;FEDERAL RESERVE PLAZA, 600 ATLANTIC AVENUE, BOSTON, MA, 02210-2211, US

(73) Assignee: U.S. Genomics, Inc., Woburn, MA (US)

(21) Appl. No.: 10/984,189

(22) Filed: Nov. 8, 2004

Related U.S. Application Data
(60) Provisional application No. 60/518,486, filed on Nov. 7, 2003.

(51) Int. Cl. C12Q 1/68; C07H 21/04

(52) U.S. Cl. 435/6; 536/24.3; 536/25.32

(57) ABSTRACT

The invention relates to methods and products for analyzing polymers using FRET. In particular the methods involve improvements in FRET signaling.

### RELATED APPLICATIONS

This application claims priority under 35 U.S.C. § 119(e) to U.S. Provisional Application Ser. No. 60/518,486, entitled “Improved FRET Efficiency Methods,” filed on Nov. 7, 2003, which is herein incorporated by reference in its entirety.

### FIELD OF THE INVENTION

The present invention relates generally to FRET based methods and related compositions for polymer analysis.

### BACKGROUND OF THE INVENTION

The study of molecular and cellular biology is focused on the microscopic structure of cells. It is known that cells have a complex microstructure that determines the functionality of the cell. Much of the diversity associated with cellular structure and function is due to the ability of a cell to assemble various building blocks into diverse chemical compounds. The cell accomplishes this task by assembling polymers from a limited set of building blocks referred to as monomers. One key to the diverse functionality of polymers is based in the primary sequence of the monomers within the polymer. This sequence is integral to understanding the basis for cellular function, such as why a cell differentiates in a particular manner or how a cell will respond to treatment with a particular drug.

The ability to identify the structure of polymers by identifying the sequence of monomers is integral to the understanding of each active component and the role that component plays within a cell. By determining the sequences of polymers it is possible to generate expression maps, to determine what proteins are expressed, to understand where mutations occur in a disease state, and to determine whether a polymer has better function or loses function when a particular monomer is absent or mutated.

Many technologies relating to genomic sequencing and analysis require site-specific labeling of nucleic acids. Most site-specific labeling is carried out using nucleic acid based probes that hybridize to their complementary sequences within a target molecule. The specificity of these probes will vary however depending upon their length, their sequence, the hybridization conditions, and the like. The ability to increase the specificity of these probes and, at the same time, use less of them would make labeling reactions more efficient and less expensive to run.

### SUMMARY OF THE INVENTION

The invention relates to methods and related compositions for polymer analysis using an improved FRET based analysis.

In one aspect, the invention provides a method for identifying a sequence of a polymer comprising contacting a polymer with two sequence specific probes capable of hybridizing to immediately adjacent sections of the polymer, wherein the probes are labeled with a fluorophore pair at their immediately adjacent terminal units, and wherein at least one member of the fluorophore pair is tethered to one of the two sequence specific probes via a linker, and detecting fluorescence or quenching from the fluorophore pair to identify the sequence of the polymer.

In one embodiment, a first member of the fluorophore pair is tethered to a first sequence specific probe via a linker and a second member of the fluorophore pair is tethered to a second sequence specific probe directly. In another embodiment, a first member of the fluorophore pair is tethered to a first sequence specific probe via a linker and a second member of the fluorophore pair is tethered to a second sequence specific via a linker.

The donor fluorophore may be tethered to a first sequence specific probe at its 5′ end and the acceptor fluorophore may be tethered to a second sequence specific probe at its 3′ end. Alternatively, the donor fluorophore may be tethered to a first sequence specific probe at its 3′ end and the acceptor fluorophore may be tethered to a second sequence specific probe at its 5′ end.

Depending upon the embodiments, the linker may be a nucleic acid linker of 2-20 nucleotides in length, or a nucleic acid linker of 5-15 nucleotides in length, or a nucleic acid linker of 5-10 nucleotides in length. Preferably the nucleotides of the linker are thymidines. The linker may also be a carbon chain.

Various embodiments apply equally to the different aspects of the invention. Some of these various embodiments are as follows. The polymer may be a nucleic acid such as a DNA or RNA, whether naturally occurring or not, although it is not so limited. The nucleic acid may be single or double stranded. The sequence specific probes may be DNA, RNA, PNA, LNA, or combinations thereof, but are not so limited. A first member of the fluorophore pair may be a donor fluorophore and a second member may be a quencher fluorophore.

A first member of the fluorophore pair may be a donor fluorophore and a second member may be an acceptor fluorophore. In one embodiment, the donor fluorophore is Cy3. In a related embodiment, the acceptor fluorophore is Cy5. In one embodiment, the donor fluorophore is tethered to a first or a second sequence specific probe via a linker. In another embodiment, the acceptor fluorophore is tethered to a first or a second sequence specific probe via a linker. In one embodiment, the donor fluorophore is tethered to a first sequence specific probe via a linker and the acceptor fluorophore is tethered to a second sequence specific probe via a linker. In another embodiment, the donor fluorophore is tethered to a first sequence specific probe directly and the acceptor fluorophore is tethered to a second sequence specific probe via a linker. In yet another embodiment, the donor fluorophore is tethered to first sequence specific probe via a linker and the acceptor fluorophore is tethered to a second sequence specific probe directly.

In another aspect, the invention provides a method for identifying a sequence of a polymer comprising contacting a polymer with two sequence specific probes capable of hybridizing to immediately adjacent sections of the polymer, wherein the probes are each tethered to a member of a fluorophore pair at a terminal unit or an internal unit, and wherein the distance between the members of the fluorophore pair is 4-22 nucleotides, and detecting fluorescence or quenching from the fluorophore pair to identify the sequence of the polymer.

In one embodiment, a first member of the fluorophore pair is tethered to a first sequence specific probe directly and a second member of the fluorophore pair is tethered to a second sequence specific probe directly. In another embodiment, a first member of the fluorophore pair is tethered to a first sequence specific probe directly and a second member of the fluorophore pair is tethered to a second sequence specific probe via a linker. In yet another embodiment, a first member of the fluorophore pair is tethered to a first sequence specific probe via a linker and a second member of the fluorophore pair is tethered to a second sequence specific probe via a linker.

A first member of the fluorophore pair may be tethered a first sequence specific probe at an internal unit and a second member of the fluorophore pair may be tethered to a second sequence specific probe at an internal unit. In another embodiment, a first member of the fluorophore pair may be tethered to a first sequence specific probe at an internal unit and a second member of the fluorophore pair may be tethered to a second sequence specific probe at a terminal unit. In yet another embodiment, a first member of the fluorophore pair is tethered to a first sequence specific probe at a terminal unit and a second member of the fluorophore pair is tethered to a second sequence specific probe at a terminal unit.

Depending on the embodiment, the distance between the members of the fluorophore pair may be 4-17 nucleotides, or 7-17 nucleotides, or 7-12 nucleotides.

In various embodiments, the distance between the members of the fluorophore pair yields at least 65% FRET efficiency.

In yet another aspect, the invention provides a composition comprising a nucleic acid probe, and a fluorophore tethered to the nucleic acid with a thymidine linker, wherein the thymidine linker is between 2-10 nucleotides in length.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

The phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

### BRIEF DESCRIPTION OF THE DRAWINGS

The figures are illustrative only and are not required for enablement of the invention disclosed herein.

FIG. 1 is a schematic diagram depicting some embodiments of the invention. The designations T10, T5 and T0 refer to linkers of 10 Ts and 5 Ts and no linker, respectively. T0sp4 refers to no linker but including a 4 nucleotide space between the hybridized probes. The linker in the diagram links the donor (Cy3 in the diagram) to the probe.

FIG. 2 is a bar graph depicting FRET output (measured as Ei) for the 4 constructs represented in FIG. 1. Fluorolog data indicate that FRET efficiency (FRET E) for the TOsp4 probe is marginally greater than the FRET E for T10 and T5.

FIG. 3 is a bar graph depicting results of single molecule counting (measured in peaks/second) for the 4 constructs represented in FIG. 1. Samples with high FRET E reveal more target molecules at a given polymer concentration, such as RNA concentration.

FIG. 4 is a bar graph depicting results of single molecule counting (measured in average peak height—number of photons) for the 4 constructs represented in FIG. 1.

FIG. 5 is a histogram depicting photon counting results for the 4 constructs represented in FIG. 1. T10, T5 and T0sp4 “tails” give similar photon counting results as E. coli spike 8 FRET donor probes. Photon counts are normalized to 100%.

### DETAILED DESCRIPTION OF THE INVENTION

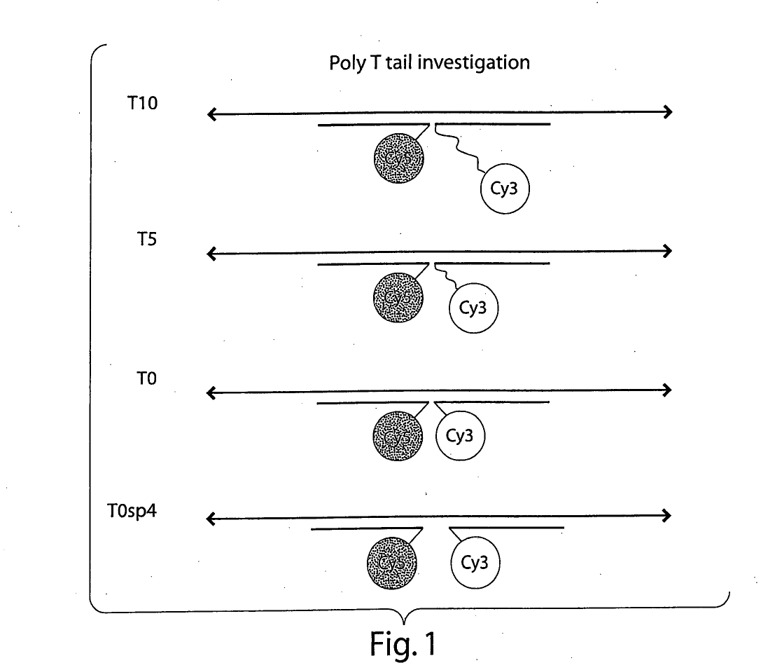

Methods for identifying information about a polymer, such as nucleotide sequence of the polymer are described. The methods involve contacting a polymer with two sequence specific probes capable of hybridizing to adjacent sections of the polymer. The probes are individually labeled with members of a fluorophore pair, such that the fluorophore pair is positioned within a specific distance or distance range when the sequence specific probes hybridize to the polymer. This may be accomplished in several ways. Two exemplary methods for accomplishing this are depicted in FIG. 1. Fluorescence or quenching from the fluorophore pair is detected to identify the sequence of or to otherwise analyze the polymer.

The members of the fluorophore pair are located at a distance in which optimal FRET signals (and thus efficiency) are achieved. The invention involves the use of two probes which hybridize to adjacent sections, and preferably immediately adjacent sections, of a target polymer. Prior art methods based on these configurations therefore positioned fluorophores in the closest possible proximity to one another. The invention provides for a greater degree of separation of the fluorophores, even in the context of probes that bind to immediately adjacent sections on the target polymer. Surprisingly the increase in separation or distance between the probes did not diminish FRET signal and in most instances actually resulted in an improved signal. An optimal range of distance between the fluorophores is 3-30 nucleotides which corresponds to 10.2 Å-102 Å. More preferably, the distance is 4-22 nucleotides which corresponds to 13.6 Å-74.8 Å. The optimal distance may be any distance value between and including the ranges listed herein, as if each and every length was explicitly recited herein. For instance, other useful ranges include but are not limited to 17 Å-34 Å, 17 Å-51 Å, 17 Å-68 Å, 17 Å-85 Å, 17 Å-102 Å, or 34 Å-68 Å. Optionally, the distance may be at least 10.2 Å, at least 13.6 Å, at least 15 Å, at least 20 Å, at least 25 Å, at least 30 Å, at least 35 Å, at least 40 Å, at least 45 Å, at least 50 Å, at least 55 Å, at least 60 Å, at least 65 Å, at least 70 Å, at least 75 Å, at least 74.8 Å, at least 80 Å, at least 85 Å, at least 90 Å, at least 95 Å, at least 100 Å, at least 105 Å, or more. In some embodiments, the distance between the units to which the fluorophores are directly or indirectly tethered is one in which, in the absence of a linker, no FRET signal would be observed due to quenching.

Energy transfer efficiency reflects the amount of excitation energy which is actually absorbed by the donor molecule and transferred to the acceptor molecule (as evidenced by the amount of emission energy produced). FRET efficiency has generally been considered to be dependent on the distance separating donor and acceptor fluorophores. FRET efficiency therefore has been used as an indicator of the distance between donor and acceptor fluorophores (and the corresponding molecules or atoms to which they are attached), with decreased FRET efficiency correlating with increased distance (i.e., an inverse correlation).

The invention provides methods in which the distance between the members of the fluorophore pair yields at least 65% FRET efficiency, more preferably at least 70% FRET efficiency, and even more preferably at least 75% FRET efficiency. It is to be understood that the invention also contemplates FRET efficiencies that are at least 80%, at least 90%, at least 95%, at least 99%, or 100%. There are a variety of ways of measuring FRET efficiency, and those of ordinary skill in the art will be familiar with such methods.

FIG. 1 is a schematic diagram of some examples of the methods of the invention. The relative position of fluorophore labeled probe sets on a target polymer is depicted in FIG. 1. Each of the 4 drawings includes a top line with arrow ends which represents the polymer being analyzed (i.e., the target). The two shorter lines refer to an exemplary probe set. The probe labeled with Cy5 in each schematic is tethered directly to the Cy5 acceptor fluorophore. It is to be understood however that the probe labeled with Cy5 in each schematic may also be tethered indirectly to the Cy5 acceptor fluorophore (e.g., via a linker). The probes are hybridized to immediately adjacent sections of the polymer in the T10, T5 and T0 schematics. Depending upon where the fluorophores are tethered to the probes (i.e., at the ends or internally), both, only one, or none of the fluorophores need to be tethered via a linker. This latter point is controlled by the distance between the units to which each fluorophore pair member is tethered.

In the T10 and T5 schematics one of the probes is tethered to a linker which is tethered at its other end to a Cy3 fluorophore. T10 and T5 refer to linkers of 10 and 5 thymidine (T) nucleotides, respectively. The schematic labeled as T0 refers to a control in which the second probe is directly tethered to a Cy3 fluorophore without the use of a linker or other spacer. In both schematics, one of the probes has a fluorophore tethered to it via a linker and the other probe has a fluorophore directly tethered to it. It is also possible for each probe to be tethered to its respective fluorophore through separate linkers, one tethered to each probe.

In the first three schematics shown in FIG. 1 the probes hybridize to immediately adjacent sections of the polymer but the fluorophore pairs are separated by a distance through the use of one or more linkers. The linkers separate the acceptor and donor fluorophore to produce suitable FRET efficiency. The fluorophores are tethered either directly or indirectly to the terminal units of the polymer.

The term “adjacent sections of the polymer” as used herein refers to two sections along the length of a polymer which are in close proximity to one another in a primary structure of the polymer. Two probes may hybridize to adjacent sections of the polymer by hybridizing to immediately adjacent sections or to spaced adjacent sections. The term “immediately adjacent sections” refers to two sections of a polymer which have no intervening units, e.g., two sections of a nucleic acid that are directly connected to one another without any intervening nucleotides. The term “spaced adjacent sections” refers to two sections of a polymer that are separated from one another by one or more units, e.g., two sections of a nucleic acid that are connected to one another by one or more intervening nucleotides. Preferably, the methods of the invention are used to detect binding of probes that hybridize to immediately adjacent sections of a polymer.

Another example (not depicted in FIG. 1) involves the use of probes that hybridize to immediately adjacent sections of the polymer, but on which fluorophores are directly or indirectly tethered to an internal unit, or a combination of internal and terminal units on the probe pair. The fluorophores may both be directly linked to the probes or indirectly linked to the probes via one or more linkers. For example, if the polymer is a nucleic acid, the fluorophores may be tethered to internal nucleotides of a nucleic acid probe, or one may be tethered to an internal nucleotide and one may be tethered to a terminal nucleotide of a nucleic acid probe. Thus, for example, the optimal distance between fluorophore pair members is the sum of the distance between the nucleotides to which the fluorophores are tethered and the length of the linkers, if there are any.

The term “terminal unit” refers to a unit at the end of the probe. The term “internal unit” refers to a unit that is positioned between the terminal units of the probe. Similarly, the term “terminal nucleotide” refers to a nucleotide at the end of the probe, i.e., a 5′ or 3′ end. The term “internal nucleotide” refers to a nucleotide that is positioned between the terminal nucleotides of the probe. “Immediately adjacent terminal units” are terminal units of probes that are positioned immediately next to each when the probes are hybridized to the polymer (i.e., there are no intervening residues between the end of one probe and the beginning of the next when both are both to the polymer).

It is to be understood that sequence information is derived from the hybridization of the sequence specific probes to the nucleic acid target. Hybridization of the sequence specific probes and their location along the length of the nucleic acid target is indicated by FRET. FRET can be detected in at least one of two ways: fluorescence or quenching. In fluorescence, a detector is set to the emission spectra of the acceptor fluorophore and binding of the sequence specific probes is indicated by energy transfer from the donor to the acceptor and fluorescence from the acceptor. In quenching, the detector is set to the emission spectra of the donor fluorophore and binding of the sequence specific probes is indicated by energy transfer from the donor to the acceptor and quenching of emission from the donor. It will be understood that minor variations of the foregoing will apply in the various aspects of the invention.

The schematic labeled T0sp4 in FIG. 1 illustrates hybridization with a second probe which is tethered directly to a Cy3 fluorophore, without a linker, but which is spaced 4 nucleotides apart from the first probe along the length of the polymer. This is an example of a probe set that hybridizes to spaced adjacent sections. In this Example the fluorophores are tethered directly to the terminal units of the probe, but the probes are spaced apart from one another when they are hybridized to create the long range FRET distance.

T10, T5 and T0sp4 constructs (shown in FIG. 1) produced similar FRET E and average intensity (of FRET peaks), all of which were significantly greater than that of T0 constructs. T10 and T5 give similar peak counts (i.e., 204 and 202, respectively).

Single molecule detection (SMD) of low FRET E samples (i.e., T0 construct) showed low relative intensity of FRET peaks and low FRET peak count. T10, T5 and T0sp4 produced a high relative FRET E, good FRET peak average intensity and higher average FRET peak count than T0 (no linker).

Thus, many embodiments of the invention require tethering of fluorophores to probes, preferably via linkers. The linker is preferably one that does not interact with itself and thus remains in a relatively linear form (i.e., no secondary structure is observed).

These spacers can be any of a variety of molecules, preferably non-active, such as nucleotides or multiple nucleotides, straight or branched saturated or unsaturated carbon chains of carbon, phospholipids, and the like, whether naturally occurring or synthetic. Additional spacers include alkyl and alkenyl carbonates, carbamates and carbamides. Abasic linkers are also contemplated.

A wide variety of spacers can be used, many of which are commercially available, for example, from sources such as Boston Probes, Inc. (now Applied Biosystems, Inc.). Spacers are not limited to organic spacers, and rather can be inorganic also (e.g., —O—Si—O—, or O—P—O—). Additionally, they can be heterogeneous in nature (e.g., composed of organic and inorganic elements). Essentially any molecule having the appropriate size restrictions and capable of being linked to a fluorophore and probe can be used as a spacer.

The length of the spacer can vary depending slightly upon the nature of the fluorophores being used, the other spacing factors described herein (such as position of linker or fluorophore along the probe and/or distance between hybridized probes) and the detection system. In some important embodiments, it has a length of not greater than 102 Å, and in some preferred embodiments, it has a length of 10.2 Å-102 Å.

In preferred embodiments the linker is comprised of one or more nucleotides. The use of a nucleic acid as a linker is particularly useful when the probes are nucleic acid, PNA or LNA probes, because of the ease of producing the probe-linker construct. Even more preferably, the linker is substantially or completely comprised of thymidines (T). It is important that the linker units do not interact with each other so that the linker does not assume secondary structure but rather remains practically linear. If two linkers are used, it is not required that they have the same length. When the linker is one or more nucleotides, a preferred linker length is 2-30 nucleotides, a more preferred length is 2-15, and an even more preferred range is 5-10 nucleotides. Those of ordinary skill in the art can determine the actual lengths corresponding to these distances based on the distance between nucleotides in a nucleic acid which is approximately 3.4 Å. Fewer nucleotides, however, may be used particularly when the use of the linker is combined with other spacing factors, the combination of which provide a higher effective FRET distance.

The methods of the invention can be used to generate unit specific information about a polymer by capturing signals arising from the labeled polymer using the devices described herein and elsewhere to manipulate the polymer. As used herein the term “unit specific information” refers to any structural information about one, some, or all of the units of the polymer. The structural information obtained by analyzing a polymer may include the identification of characteristic properties of the polymer which (in turn) allows, for example, for the identification of the presence of a polymer in a sample, determination of the relatedness of polymers, identification of the size of the polymer, identification of the proximity or distance between two or more individual units or unit specific markers of a polymer, identification of the order of two or more individual units or unit specific markers within a polymer, and/or identification of the general composition of the units or unit specific markers of the polymer. Since the structure and function of biological molecules are interdependent, the structural information can reveal important information about the function of the polymer.

The term “analyzing a polymer” as used herein means obtaining some information about the structure of the polymer such as its size, the order of its units, its relatedness to other polymers, the identity of its units, or its presence or absence in a sample. For example, the entire or portions of the entire sequence of the polymer, the order of probes, or the time of separation between signals as an indication of the distance between the units or unit specific markers.

A “polymer” as used herein is a compound having a linear backbone of individual units which are linked together. The polymer being analyzed and/or labeled is referred to as the polymer target. In some cases, the backbone of the polymer may be branched. Preferably the backbone is unbranched. The term “backbone” is given its usual meaning in the field of polymer chemistry. The polymers may be heterogeneous in backbone composition thereby containing any possible combination of polymer units linked together. In one embodiment the polymers are, for example, nucleic acids, polypeptides, polysaccharides, or carbohydrates. In the most preferred embodiments, the polymer is a nucleic acid or a polypeptide. A polypeptide as used herein is a biopolymer comprised of linked amino acids.

The term “nucleic acid” is used herein to mean multiple linked nucleotides (i.e., molecules comprising a sugar (e.g., ribose or deoxyribose) linked to an exchangeable organic base, which is either a substituted pyrimidine (e.g., cytosine (C), thymidine (T) or uracil (U)) or a substituted purine (e.g., adenine (A) or guanine (G)). “Nucleic acid” and “nucleic acid molecule” are used interchangeably and refer to oligoribonucleotides as well as oligodeoxyribonucleotides. The terms shall also include polynucleosides (i.e., a polynucleotide minus a phosphate) and any other organic base containing polymer. The nucleic acid being analyzed and/or labeled is referred to as the nucleic acid target.

Nucleic acid targets and nucleic acid probes may be DNA or RNA, although they are not so limited. DNA may be genomic DNA such as nuclear DNA or mitochondrial DNA. RNA may be mRNA, mRNA, rRNA and the like. Nucleic acids may be naturally occurring such as those recited above, or may be synthetic such as cDNA.

Thus, nucleic acids can be obtained from existing nucleic acid sources (e.g., genomic or cDNA), or by synthetic means (e.g., produced by nucleic acid synthesis).

Harvest and isolation of nucleic acids are routinely performed in the art and suitable methods can be found in standard molecular biology textbooks. The nucleic acid may be harvested from a biological sample such as a tissue or a biological fluid. The term “tissue” as used herein refers to both localized and disseminated cell populations including but not limited, to brain, heart, breast, colon, bladder, uterus, prostate, stomach, testis, ovary, pancreas, pituitary gland, adrenal gland, thyroid gland, salivary gland, mammary gland, kidney, liver, intestine, spleen, thymus, bone marrow, trachea, and lung. Biological fluids include saliva, sperm, serum, plasma, blood and urine, but are not so limited. Both invasive and non-invasive techniques can be used to obtain such samples and are well documented in the art.

The methods of the invention may be performed in the absence of prior nucleic acid amplification in vitro. In some preferred embodiments, the nucleic acid is directly harvested and isolated from a biological sample (such as a tissue or a cell culture), without its amplification. Accordingly, some embodiments of the invention involve analysis of “non in vitro amplified nucleic acids”. As used herein, a “non in vitro amplified nucleic acid” refers to a nucleic acid that has not been amplified in vitro using techniques such as polymerase chain reaction or recombinant DNA methods.

A non in vitro amplified nucleic acid may, however, be a nucleic acid that is amplified in vivo (e.g., in the biological sample from which it was harvested) as a natural consequence of the development of the cells in the biological sample. This means that the non in vitro nucleic acid may be one which is amplified in vivo as part of gene amplification, which is commonly observed in some cell types as a result of mutation or cancer development.

In some embodiments, the invention embraces nucleic acid derivatives as targets and/or probes. As used herein, a “nucleic acid derivative” is a non-naturally occurring nucleic acid. Nucleic acid derivatives may contain non-naturally occurring elements such as non-naturally occurring nucleotides and non-naturally occurring backbone linkages. These include substituted purines and pyrimidines such as C-5 propyne modified bases, 5-methylcytosine, 2-aminopurine, 2-amino-6-chloropurine, 2,6-diaminopurine, hypoxanthine, 2-thiouracil and pseudoisocytosine. Other such modifications are well known to those of skill in the art.

The nucleic acids may also encompass substitutions or modifications, such as in the bases and/or sugars. For example, they include nucleic acids having backbone sugars which are covalently attached to low molecular weight organic groups other than a hydroxyl group at the 3′ position and other than a phosphate group at the 5′ position. Thus, modified nucleic acids may include a 2′-O-alkylated ribose group. In addition, modified nucleic acids may include sugars such as arabinose instead of ribose.

The nucleic acids may be heterogeneous in backbone composition thereby containing any possible combination of nucleic acid units linked together such as peptide nucleic acids (which have amino acid linkages with nucleic acid bases, and which are discussed in greater detail herein). In some embodiments, the nucleic acids are homogeneous in backbone composition.

As used herein with respect to linked units of a polymer, “linked” or “linkage” means two entities bound to one another by any physicochemical means. Any linkage known to those of ordinary skill in the art, covalent or non-covalent, is embraced. Natural linkages, which are those ordinarily found in nature connecting the individual units of a particular polymer, are most common. Natural linkages include, for instance, amide, ester and thioester linkages. The individual units of a polymer analyzed by the methods of the invention may be linked, however, by synthetic or modified linkages. Polymers where the units are linked by covalent bonds will be most common but those that include hydrogen bonded units are also embraced by the invention.

The polymer is made up of a plurality of individual units. An “individual unit” as used herein is a building block or monomer which can be linked directly or indirectly to other building blocks or monomers to form a polymer. The polymer preferably is a polymer of at least two different linked units.

The polymers are analyzed using probe sets that are labeled with fluorophore pairs. A fluorophore or fluorescent label is a substance which is capable of exhibiting fluorescence within a detectable range. Fluorophores include, but are not limited to, fluorescein, isothiocyanate, fluorescein amine, eosin, rhodamine, dansyl, umbelliferone, 5-carboxyfluorescein (FAM), 2′7′-dimethoxy-4′5′-dichloro-6-carboxyfluorescein (JOE), rhodamine, 6 carboxyrhodamine (R6G), N,N,N′,N′-tetramethyl-6-carboxyrhodamine (TAMRA), 6-carboxy-X-rhodamine (ROX), 4-(4′-dimethylaminophenylazo) benzoic acid (DABCYL), 5-(2′-aminoethyl) aminonaphthalene-1-sulfonic acid (EDANS), 4-acetamido-4′-isothiocyanatostilbene-2,2′disulfonic acid, acridine, acridine isothiocyanate, r-amino-N->3-vinylsulfonyl)phenyl!naphthalimide-3,5, disulfonate (Lucifer Yellow VS), N-(4-anilino-1-naphthyl)maleimide, anthranilamide, Brilliant Yellow, coumarin, 7-amino-4-methylcoumarin, 7-amino-4-trifluoromethylcouluarin (Coumaran 151), cyanosine, 4′, 6-diaminidino-2-phenylindole (DAPI), 5′,5″-diaminidino-2-phenylindole (DAPI), 5′,5″-dibromopyrogallol-sulfonephthalein (Bromopyrogallol Red), 7-diethylamino-3-(4′-isothiocyanatophenyl)-4-methylcoumarin diethylenetriamine pentaacetate, 4,4′-diisothiocyanatodihydro-stilbene-2,2′-disulfonic acid, 4,4′-diisothiocyanatostilbene-2,2′-disulfonic acid, 4-dimethylaminophenylazophenyl-4′-isothiocyanate (DABITC), eosin isothiocyanate, erythrosin B, erythrosin isothiocyanate, ethidium, 5-(4,6-dichlorotriazin-2-yl) aminofluorescein (DTAF), QFITC (XRITC), fluorescamine, IR144, IR1446, Malachite Green isothiocyanate, 4-methylumbelliferone, ortho cresolphthalein, nitrotyrosine, pararosaniline, Phenol Red, B-phycoerythrin, o-phthaldialdehyde, pyrene, pyrene butyrate, succinimidyl 1-pyrene butyrate, Reactive Red 4 (Cibacron. RTM. Brilliant Red 3B-A), lissamine rhodamine B sulfonyl chloride, rhodamine B, rhodanine 123, rhodamine X isothiocyanate, sulforhodamine B, sulforhodamine 101, sulfonyl chloride derivative of sulforhodamine 101, (Texas Red), tetramethyl rhodamine, tetramethyl rhodamine isothiocyanate (TRITC), riboflavin, rosolic acid, and terbium chelate derivatives.

Fluorophore pairs are two fluorophores that are capable of undergoing FRET to produce or eliminate a detectable signal when positioned in proximity to one another. Examples of donors include Ha10TAlexa488, Ha10TAlexa546, Ha₁₀TBODIPY493, Ha10TOysterS56, Ha10TFluor (FAM), Ha10TCy3, and HA10TTR (Tamra). Examples of acceptors include HACy5, HaAlexa594, HAAlexa647, and HaOyster656.

Fluorescence may be measured using a fluorometer. The optical emission from the fluorescence molecule, whether donor or acceptor, can be detected by the fluorometer and processed as a signal. When fluorescence is being measured in a sample fixed to various portions of a surface (e.g., when the nucleic acid is fixed), the surface can be moved using a multi-access translation stage in order to position the different areas of the surface, such that the signal can be collected. When the fluorescence is measured in solution other methods can be used for detecting the signal including the linear analysis methods described herein. Many types of fluorometers have been developed. For instance, an example of an instrument for measuring FRET is described in U.S. Pat. No. 5,911,952.

The polymer is labeled with one or more sequence specific probes. “Sequence specific” when used in the context of a nucleic acid probe means that the probe recognizes a particular linear arrangement of nucleotides or derivatives thereof. In non-nucleic acid polymers, the sequence specific probe is one that binds to a region of the polymer in a sequence specific manner, for example, by recognizing and binding to a linear arrangement of amino acids if the polymer is a peptide or protein. In preferred embodiments, the linear arrangement includes contiguous nucleotides or derivatives thereof that each bind to a corresponding complementary nucleotide on the nucleic acid target. In some embodiments, however, the sequence may not be contiguous as there may be one, two, or more nucleotides that do not have corresponding complementary residues on the target.

It is to be understood that any nucleic acid analog that is capable of recognizing a nucleic acid with structural or sequence specificity can be used as or in a nucleic acid probe. In most instances, the nucleic acid probes will form at least a Watson-Crick bond with the nucleic acid target. In other instances, the nucleic acid probe can form a Hoogsteen bond with the nucleic acid target, thereby forming a triplex. A nucleic acid sequence that binds by Hoogsteen binding enters the major groove of a nucleic acid target and hybridizes with the bases located there. Examples of these latter probes include molecules that recognize and bind to the minor and major grooves of nucleic acids (e.g., some forms of antibiotics). In some embodiments, the nucleic acid probes can form both Watson-Crick and Hoogsteen bonds with the target. Bis PNA probes, for instance, are capable of both Watson-Crick and Hoogsteen binding to a nucleic acid target.

The nucleic acid probe may be a peptide nucleic acid (PNA), a bis PNA clamp, a pseudocomplementary PNA, a locked nucleic acid (LNA), DNA, RNA, or co-polymers of the above such as DNA-LNA co-polymers. In some instances, the nucleic acid target can also be comprised of any other these elements.

PNAs are DNA analogs having their phosphate backbone replaced with 2-aminoethyl glycine residues linked to nucleotide bases through glycine amino nitrogen and methylenecarbonyl linkers. PNAs can bind to both DNA and RNA targets by Watson-Crick base pairing, and in so doing form stronger hybrids than would be possible with DNA or RNA based probes.

PNAs are synthesized from monomers connected by a peptide bond (Nielsen, P. E. et al. Peptide Nucleic Acids, Protocols and Applications, Norfolk: Horizon Scientific Press, p. 1-19 (1999)). They can be built with standard solid phase peptide synthesis technology. PNA chemistry and synthesis allows for inclusion of amino acids and polypeptide sequences in the PNA design. For example, lysine residues can be used to introduce positive charges in the PNA backbone. All chemical approaches available for the modifications of amino acid side chains are directly applicable to PNAs.

PNA has a charge-neutral backbone, and this attribute leads to fast hybridization rates of PNA to DNA (Nielsen, P. E. et al. Peptide Nucleic Acids Protocols and Applications, Norfolk: Horizon Scientific Press, p. 1-19 (1999)). The hybridization rate can be further increased by introducing positive charges in the PNA structure, such as in the PNA backbone or by addition of amino acids with positively charged side chains (e.g., lysines). PNA can form a stable hybrid with DNA molecule. The stability of such a hybrid is essentially independent of the ionic strength of its environment (Orum, H. et al., BioTechniques 19(3):472-480 (1995)), most probably due to the uncharged nature of PNAs. This provides PNAs with the versatility of being used in vivo or in vitro. However, the rate of hybridization of PNAs that include positive charges is dependent on ionic strength, and thus is lower in the presence of salt.

Several types of PNA designs exist, and these include single strand PNA (ssPNA), bis PNA and pseudocomplementary PNA (pcPNA).

The structure of PNA/DNA complex depends on the particular PNA and its sequence. Single stranded PNA (ssPNA) binds to single stranded DNA (ssDNA) preferably in antiparallel orientation (i.e., with the N-terminus of the ssPNA aligned with the 3′ terminus of the ssDNA) and with a Watson-Crick pairing. PNA also can bind to DNA with a Hoogsteen base pairing, and thereby forms triplexes with double stranded DNA (dsDNA) (Wittung, P. et al., Biochemistry 36:7973 (1997)).

Single strand PNA is the simplest of the PNA molecules. This PNA form interacts with nucleic acids to form a hybrid duplex via Watson-Crick base pairing. The duplex has different spatial structure and higher stability than dsDNA (Nielsen, P. E. et al. Peptide Nucleic Acids, Protocols and Applications, Norfolk: Horizon Scientific Press, p. 1-19 (1999)). However, when different concentration ratios are used and/or in presence of complimentary DNA strand, PNA/DNA/PNA or PNA/DNA/DNA triplexes can also be formed (Wittung, P. et al., Biochemistry 36:7973 (1997)). The formation of duplexes or triplexes additionally depends upon the sequence of the PNA. Thymine-rich homopyrimidine ssPNA forms PNA/DNA/PNA triplexes with dsDNA targets where one PNA strand is involved in Watson-Crick antiparallel pairing and the other is involved in parallel Hoogsteen pairing. Cytosine-rich homopyrimidine ssPNA preferably binds through Hoogsteen pairing to dsDNA forming a PNA/DNA/DNA triplex. If the ssPNA sequence is mixed, it invades the dsDNA target, displaces the DNA strand, and forms a Watson-Crick duplex. Polypurine ssPNA also forms triplex PNA/DNA/PNA with reversed Hoogsteen pairing.

Bis PNA includes two strands connected with a flexible linker. One strand is designed to hybridize with DNA by a classic Watson-Crick pairing, and the second is designed to hybridize with a Hoogsteen pairing. The target sequence can be short (e.g., 8 bp), but the bis PNA/DNA complex is still stable as it forms a hybrid with twice as many (e.g., a 16 bp) base pairings overall. The bis PNA structure further increases specificity of their binding. As an example, binding to an 8 bp site with a probe having a single base mismatch results in a total of 14 bp rather than 16 bp.

Preferably, bis PNAs have homopyrimidine sequences, and even more preferably, cytosines are protonated to form a Hoogsteen pair to a guanosine. Therefore, bis PNA with thymines and cytosines is capable of hybridization to DNA only at pH below 6.5. The first restriction—homopyrimidine sequence only—is inherent to the mode of bis PNA binding. Pseudoisocytosine (J) can be used in the Hoogsteen strand instead of cytosine to allow its hybridization through a broad pH range (Kuhn, H., J. Mol. Biol. 286:1337-1345 1999)).

Bis PNAs have multiple modes of binding to nucleic acids (Hansen, G. I. et al., J. Mol. Biol. 307(1):67-74 (2001)). One isomer includes two bis PNA molecules instead of one. It is formed at higher bis PNA concentration and has a tendency to rearrange into the complex with a single bis PNA molecule. Other isomers differ in positioning of the linker around the target DNA strands. All the identified isomers still bind to the same binding site/target.

Pseudocomplementary PNA (pcPNA) (Izvolsky, K. I. et al., Biochemistry 10908-10913 (2000)) involves two single stranded PNAs added to dsDNA. One pcPNA strand is complementary to the target sequence, while the other is complementary to the displaced DNA strand. As the PNA/DNA duplex is more stable, the displaced DNA generally does not restore the dsDNA structure. The PNA/PNA duplex is more stable than the DNA/PNA duplex and the PNA components are self-complementary because they are designed against complementary DNA sequences. Hence, the added PNAs would rather hybridize to each other. To prevent the self-hybridization of pcPNA units, modified bases are used for their synthesis including 2,6-diamiopurine (D) instead of adenine and 2-thiouracil (^{S}U) instead of thymine. While D and ^{S}U are still capable of hybridization with T and A respectively, their self-hybridization is sterically prohibited.

Locked nucleic acid (LNA) molecules form hybrids with DNA, which are at least as stable as PNA/DNA hybrids (Braasch, D. A. et al., Chem & Biol. 8(1):1-7(2001)). Therefore, LNA can be used just as PNA molecules would be. LNA binding efficiency can be increased in some embodiments by adding positive charges to it. LNAs have been reported to have increased binding affinity inherently.

Commercial nucleic acid synthesizers and standard phosphoramidite chemistry are used to make LNAs. Therefore, production of mixed LNA/DNA sequences is as simple as that of mixed PNA/peptide sequences. The stabilization effect of LNA monomers is not an additive effect. The monomer influences conformation of sugar rings of neighboring deoxynucleotides shifting them to more stable configurations (Nielsen, P. E. et al. Peptide Nucleic Acids Protocols and Applications, Norfolk: Horizon Scientific Press, p. 1-19 (1999)). Also, lesser number of LNA residues in the sequence dramatically improves accuracy of the synthesis. Naturally, most of biochemical approaches for nucleic acid conjugations are applicable to LNA/DNA constructs.

The probes can also be stabilized in part by the use of other backbone modifications. The invention intends to embrace, in addition to the peptide and locked nucleic acids discussed herein, the use of the other backbone modifications such as but not limited to phosphorothioate linkages, phosphodiester modified nucleic acids, combinations of phosphodiester and phosphorothioate nucleic acid, methylphosphonate, alkylphosphonates, phosphate esters, alkylphosphonothioates, phosphoramidates, carbamates, carbonates, phosphate triesters, acetamidates, carboxymethyl esters, methylphosphorothioate, phosphorodithioate, p-ethoxy, and combinations thereof.

Other backbone modifications, particularly those relating to PNAs, include peptide and amino acid variations and modifications. Thus, the backbone constituents of PNAs may be peptide linkages, or alternatively, they may be non-peptide linkages. Examples include acetyl caps, amino spacers such as O-linkers, amino acids such as lysine (particularly useful if positive charges are desired in the PNA), and the like. Various PNA modifications are known and probes incorporating such modifications are commercially available from sources such as Boston Probes, Inc.

One limitation of the stability of nucleic acid hybrids is the length of the probe, with longer probes leading to greater stability than shorter probes. Notwithstanding this proviso, the probes of the invention can be any length ranging from at least 4 nucleotides long to in excess of 1000 nucleotides long. In preferred embodiments, the probes are 5-100 nucleotides in length, more preferably between 5-25 nucleotides in length, and even more preferably 5-12 nucleotides in length. The length of the probe can be any length of nucleotides between and including the ranges listed herein, as if each and every length was explicitly recited herein. It should be understood that not all residues of the probe need hybridize to complementary residues in the nucleic acid target. For example, the probe may be 50 residues in length, yet only 25 of those residues hybridize to the nucleic acid target. Preferably, the residues that hybridize are contiguous with each other.

The probes are preferably single stranded, but they are not so limited. For example, when the probe is a bis PNA it can adopt a secondary structure with the nucleic acid target resulting in a triple helix conformation, with one region of the bis PNA clamp forming Hoogsteen bonds with the backbone of the target and another region of the bis PNA clamp forming Watson-Crick bonds with the nucleotide bases of the target.

The nucleic acid probe hybridizes to a complementary sequence within the nucleic acid target. The specificity of binding can be manipulated based on the hybridization conditions. For example, salt concentration and temperature can be modulated in order to vary the range of sequences recognized by the nucleic acid probes.

Other probe sets include antibodies or antibody fragments and their corresponding antigen or hapten binding partners. Detection of such bound antibodies and proteins or peptides is accomplished by techniques well known to those skilled in the art. Antibody/antigen complexes are easily detected by linking a fluorophore to the antibodies which recognize the polymer and then observing the site of the label. Polyclonal and monoclonal antibodies may be used. Antibody fragments include Fab, F(ab)₂, Fd and antibody fragments which include a CDR3 region.

The polymers may be analyzed using a single molecule analysis system (e.g., a single polymer analysis system). A single molecule detection system is capable of analyzing single molecules separately from other molecules. Such a system may be capable of analyzing single molecules either in a linear manner (i.e., starting at a point and then moving progressively in one direction or another) and/or, as may be more appropriate in the present invention, in their totality. In certain embodiments in which detection is based predominately on the presence or absence of a signal, linear analysis may not be required. However, there are other embodiments embraced by the invention which would benefit from the ability to linearly analyze molecules (preferably polymers) in a sample. These include applications in which the sequence of the polymer is desired.

A linear polymer analysis system is a system that analyzes polymers in a linear manner (i.e., starting at one location on the polymer and then proceeding linearly in either direction therefrom). As a polymer is analyzed, the detectable labels attached to it are detected in either a sequential or simultaneous manner. When detected simultaneously, the signals usually form an image of the polymer, from which distances between labels can be determined. When detected sequentially, the signals are viewed in histogram (signal intensity vs. time), that can then be translated into a map, with knowledge of the velocity of the polymer. It is to be understood that in some embodiments, the polymer is attached to a solid support, while in others it is free flowing. In either case, the velocity of the polymer as it moves past, for example, an interaction station or a detector, will aid in determining the position of the labels, relative to each other and relative to other detectable markers that may be present on the polymer.

Accordingly, the analysis systems useful in the invention may deduce the total amount of label on a polymer, and in some instances, the location of such labels. The ability to locate and position the labels allows these patterns to be superimposed on other genetic maps, in order to orient and/or identify the regions of the genome being analyzed.

An example of a suitable system is the GeneEngine™ (U.S. Genomics, Inc., Woburn, Mass.). The Gene Engine™ system is described in PCT patent applications WO98/35012 and WO00/09757, published on Aug. 13, 1998, and Feb. 24, 2000, respectively, and in issued U.S. Pat. No. 6,355,420 B1, issued Mar. 12, 2002. The contents of these applications and patent, as well as those of other applications and patents, and references cited herein are incorporated by reference in their entirety. This system is both a single molecule analysis system and a linear polymer analysis system. It allows single nucleic acids to be passed through an interaction station in a linear manner, whereby the nucleotides in the nucleic acids are interrogated individually in order to determine whether there is a detectable label conjugated to the nucleic acid. Interrogation involves exposing the nucleic acid to an energy source such as optical radiation of a set wavelength. In response to the energy source exposure, the detectable label on the nucleotide emits a signal which is exposed to the second fluorophore of the fluorophore pair (if present in the vicinity) to produce a detectable signal. The mechanism for signal emission and detection will depend on the type of label sought to be detected.

Other single molecule nucleic acid analytical methods which involve elongation of DNA molecules can also be used in the methods of the invention. These include fiber-fluorescence in situ hybridization (fiber-FISH) (Bensimon, A. et al., Science 265(5181):2096-2098 (1997)). In fiber-FISH, nucleic acids are elongated and fixed on a surface by molecular combing. Hybridization with fluorescently labeled probe sequences allows determination of sequence landmarks on the nucleic acid molecules. The method requires fixation of elongated molecules so that molecular lengths and/or distances between markers can be measured. Pulse field gel electrophoresis can also be used to analyze the labeled nucleic acids. Pulse field gel electrophoresis is described by Schwartz, D. C. et al., Cell 37(1):67-75 (1984). Other nucleic acid analysis systems are described by Otobe, K. et al., Nucleic Acids Res. 29(22):E109 (2001), Bensimon, A. et al. in U.S. Pat. No. 6,248,537, issued Jun. 19, 2001, Herrick, J. et al., Chromosome Res. 7(6):409:423 (1999), Schwartz in U.S. Pat. No. 6,150,089 issued Nov. 21, 2000 and U.S. Pat. No. 6,294,136, issued Sep. 25, 2001. Other linear polymer analysis systems can also be used, and the invention is not intended to be limited to solely those listed herein.

Optical detectable signals are generated, detected and stored in a database. The signals can be analyzed to determine structural information about the polymer. The signals can be analyzed by assessing the intensity of the signal to determine structural information about the polymer. The computer may be the same computer used to collect data about the polymers, or may be a separate computer dedicated to data analysis. A suitable computer system to implement embodiments of the present invention typically includes an output device which displays information to a user, a main unit connected to the output device and an input device which receives input from a user. The main unit generally includes a processor connected to a memory system via an interconnection mechanism. The input device and output device also are connected to the processor and memory system via the interconnection mechanism. Computer programs for data analysis of the detected signals are readily available from CCD (charge coupled device) manufacturers.

The present invention is further illustrated by the following Examples, which in no way should be construed as further limiting.

### EXAMPLES

### Example 1

### Analysis of T10, T5, T0, and T0sp4* as Cy3 Donor Probes on E. coli Spike 8 (2 kb) Target RNA

Materials and Methods:

Sample preparation: 50 nM sRNA (E. Coli spike 8) was probed with 50 nM cDNA probes by overnight hybridization (70C, 10 minutes; 55C, overnight). The samples were diluted to 2 nM in TRIS (10 mM pH 8.5) to run on Fluorolog3 and to 50 pM in TRIS (10 mM pH 8.5) to run on DCP/sipper (50 μm ID square).

A schematic diagram of the probes and their relative position on the E. coli sRNA is depicted in FIG. 1. Each of the 4 drawings includes a top line with arrow ends which refers to the E. coli spike 8 sRNA. The two shorter lines refer to the two probes used in each study. The probe labeled with Cy5 in each schematic is tethered directly to the Cy5 acceptor fluorophore. The schematic labeled T10 refers to hybridization with a second probe having a linker of 10 T nucleotides, which is tethered on the other end to a Cy3 fluorophore. The schematic labeled T5 to hybridization with a second probe having a linker of 5 T nucleotides, which is tethered on the other end to a Cy3 fluorophore. The schematic labeled as T0 refers to a control and refers to hybridization with a second probe which is directly tethered to a Cy3 fluorophore without the use of a linker or other spacer. The schematic labeled T0sp4 refers to hybridization with a second probe which is tethered directly to a Cy3 fluorophore, without a linker, but which is spaced 4 nucleotides apart from the first probe along the length of the RNA.

Results:

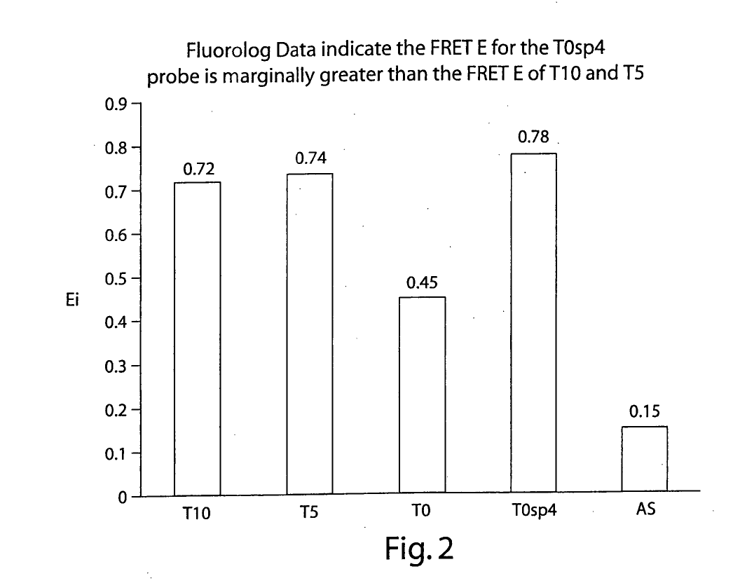

The results are shown in FIGS. 2-5. A summary of the results is also listed in Table 1 below.

FIG. 2 is a bar graph depicting Fluorolog Data indicating the FRET E for the three probe sets utilizing a long range FRET distance is greater than the probe set using traditional FRET distances (T0). T0sp4 probe was marginally greater than the FRET E of T10 and T5.

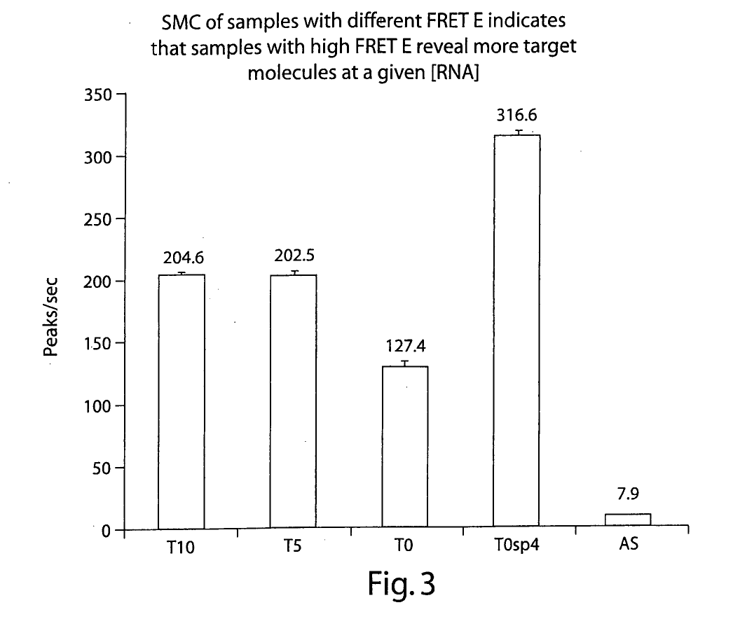

FIG. 3 is a bar graph depicting data as peaks per second based on single molecule counting (SMC). SMC of samples with different FRET E demonstrated that samples with high FRET E reveal more target molecules at a given RNA concentration. Thus, the three probe sets utilizing a long range FRET distance identified more target molecules than the probe set using traditional FRET distances (T0).

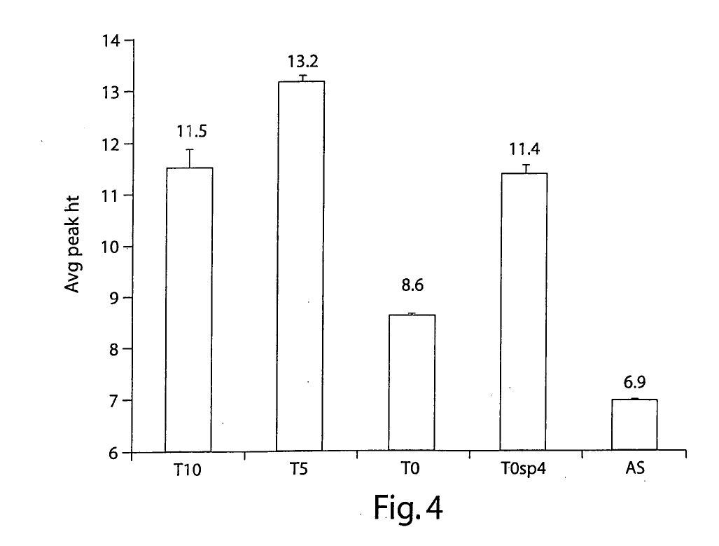

FIG. 4 is a bar graph depicting the number of photons counted as measured by average peak height based on single molecule counting (SMC). SMC of samples with different FRET E demonstrated that samples with high FRET E also have higher average intensity. Thus, the three probe sets utilizing a long range FRET distance have higher intensity than the probe set using traditional FRET distances (T0).

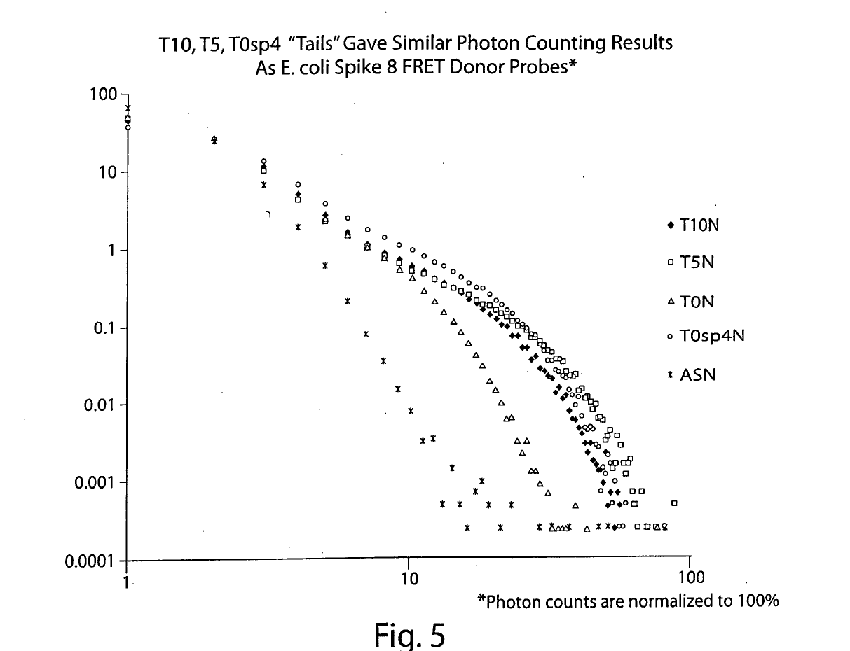

FIG. 5 depicts a histogram which reveals the photons counts per molecule. The data demonstrates that probe sets T10, T5, and T0sp4 “Tails” produced similar photon counting results as E. coli Spike 8 FRET donor probes.

TABLE 1

| | FRET E | FRET | Average | |
|---|---|---|---|---|
| Probe | (Fluorolog) | Peaks/s | Intensity | Notes |
| T10 | 0.72 | 204.65 | 11.53 | Th based on asRNA |
| T5 | 0.74 | 202.46 | 13.16 | Th based on asRNA |
| T0 | 0.45 | 127.37 | 8.6 | Th based on asRNA |
| T0sp4 | 0.78 | 316.61 | 11.43 | Th based on asRNA |
| asRNA | 0.15 | 7.87 | 6.94 | [noise] + 5σ |

### Equivalents

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

The entire contents of all of the references (including literature references, issued patents, published patent applications, and co-pending patent applications) cited throughout this application are hereby expressly incorporated by reference.

1. A method for identifying a sequence of a polymer comprising:
contacting a polymer with two sequence specific probes capable of hybridizing to immediately adjacent sections of the polymer, wherein the probes are labeled with a fluorophore pair at their immediately adjacent terminal units, and wherein at least one member of the fluorophore pair is tethered to one of the two sequence specific probes via a linker, and detecting fluorescence or quenching from the fluorophore pair to identify the sequence of the polymer.

2. The method of claim 1, wherein the polymer is a nucleic acid.

3. The method of claim 2, wherein the nucleic acid is DNA or RNA.

4. The method of claim 1, wherein a first member of the fluorophore pair is tethered to a first sequence specific probe via a linker and a second member of the fluorophore pair is tethered to a second sequence specific probe directly.

5. The method of claim 1, wherein a first member of the fluorophore pair is tethered to a first sequence specific probe via a linker and a second member of the fluorophore pair is tethered to a second sequence specific via a linker.

6. The method of claim 1, wherein a first member of the fluorophore pair is a donor fluorophore and a second member is an acceptor fluorophore.

7. The method of claim 1, wherein a first member of the fluorophore pair is a donor fluorophore and a second member is a quencher fluorophore.

8. The method of claim 6, wherein the donor fluorophore is tethered to a first or a second sequence specific probe via a linker.

9. The method of claim 6, wherein the acceptor fluorophore is tethered to a first or a second sequence specific probe via a linker.

10. The method of claim 6, wherein the donor fluorophore is tethered to a first sequence specific probe via a linker and the acceptor fluorophore is tethered to a second sequence specific probe via a linker.

11. The method of claim 6, wherein the donor fluorophore is tethered to a first sequence specific probe directly and the acceptor fluorophore is tethered to a second sequence specific probe via a linker.

12. The method of claim 6, wherein the donor fluorophore is tethered to first sequence specific probe via a linker and the acceptor fluorophore is tethered to a second sequence specific probe directly.

13. The method of claim 6, wherein the donor fluorophore is tethered to a first sequence specific probe at its 5′ end and the acceptor fluorophore is tethered to a second sequence specific probe at its 3′ end.

14. The method of claim 6, wherein the donor fluorophore is tethered to a first sequence specific probe at its 3′ end and the acceptor fluorophore is tethered to a second sequence specific probe at its 5′ end.

15. The method of claim 4, wherein the linker is a nucleic acid linker of 2-20 nucleotides in length.

16. The method of claim 4, wherein the linker is a nucleic acid linker of 5-15 nucleotides in length.

17. The method of claim 4, wherein the linker is a nucleic acid linker of 5-10 nucleotides in length.

18. The method of claim 4, wherein the linker is a nucleic acid linker of 2-20, 5-15 or 5-10 thymidines in length.

19-21. (canceled)

22. A method for identifying a sequence of a polymer comprising:
contacting a polymer with two sequence specific probes capable of hybridizing to immediately adjacent sections of the polymer, wherein the probes are each tethered to a member of a fluorophore pair at a terminal unit or an internal unit, and wherein the distance between the members of the fluorophore pair is 4-22 nucleotides, and detecting fluorescence or quenching from the fluorophore pair to identify the sequence of the polymer.

23-31. (canceled)

32. A composition comprising:
a nucleic acid probe, and a fluorophore tethered to the nucleic acid with a thymidine linker, wherein the thymidine linker is between 2-10 nucleotides in length.
